(19) United States
(12) Patent Application Publication
(10) Pub. No.: US 2019/0000676 A1
(43) Pub. Date: Jan. 3, 2019

(54) ABSORBENT SHEET

(71) Applicant: Oji Holdings Corporation, Tokyo (JP)

(72) Inventors: Ayako SHIRAI, Tokyo (JP); Takashi YAEZAWA, Fuji-shi, Shizuoka (JP); Mariko SUGAYA, Tokyo (JP); Asami KAWAGUCHI, Tokyo (JP); Yuko SAITO, Tokyo (JP)

(21) Appl. No.: 15/741,364

(22) PCT Filed: Jun. 29, 2016
(86) PCT No.: PCT/JP2016/069292

§ 371 Date: Jan. 2, 2018

(30) Foreign Application Priority Data
Jun. 30, 2015 (JP) 2015-131629

(51) Int. Cl. A61F 13/00; B01J 20/24; B01J 20/26; B01J 20/28; A61F 13/02; A61L 15/60; A61L 15/22; A61L 15/46; A61L 15/38; A61L 15/40; A61L 15/18; A61L 15/58

(52) U.S. Cl. CPC A61F 13/00063; A61F 2013/00604; B01J 20/261; B01J 20/28038; A61F 13/0256; A61F 13/00991; A61F 13/0206; A61L 15/60; A61L 15/225; A61L 15/46; A61L 15/38; A61L 15/40; A61L 15/18; A61L 15/58; A61F 2013/00719; B01J 20/24

(57) ABSTRACT

The purpose of the present invention is to provide an absorbent sheet capable of sufficiently absorbing exudates exuding from an area of a lesion and exhibiting a powerful deodorizing effect for illness-associated odors. The invention relates to an absorbent sheet with deodorizing functions having an absorbent region and a deodorizing region situated to at least one side of the absorbent region, wherein the absorbent region contains cellulosic fibers, an absorbent polymer and a thermal adhesive resin, and the deodorizing region contains cellulosic fibers, a deodorizing agent and a thermal adhesive resin.

### TECHNICAL FIELD

The present invention relates to an absorbent sheet. Specifically, the present invention relates to an absorbent sheet with deodorizing functions that has an absorbent region and a deodorizing region.

### BACKGROUND ART

Heretofore, it is known that a lesion in the skin may often give off distinctive odors (hereinafter this may be referred to as illness-associated odors). In particular, it is known that patients with advanced cancer by invasion in the skin from breast cancer, skin cancer, head and neck cancer and the like may give off sulfurous distinctive, illness-associated odors. When having strong odors associated with illness, patients may often worry about the odors and may have a risk of isolation as refusing to meet with other persons. In addition, medical service workers and patients' families must carry out nursing care in an environment of strong illness-associated odors, and may often assume a heavy burden.

As a measure for relieving such illness-associated odors, a lesion to give off illness-associated odors may be covered with a covering material. For example, PTL 1 discloses an illness odors-suppressing sheet for medical use having a nonwoven fabric infiltrated with a deodorant. In this, a deodorizing effect is expected by using a nonwoven fabric infiltrated with a deodorant. PTL 2 discloses a wound covering material having an absorbent layer. The wound covering material disclosed in PTL 2 is mainly to protect a wound region and to absorb a wound exudate, and also to prevent odor generation from a wound region. In PTL 2, the wound covering material has an activated carbon fabric layer in addition- to the absorbent layer therein, thereby proposing absorption of the exudates and the odors from a wound region.

### CITATION LIST

### Patent Literature

PTL 1: JP 2014-113289 A

PTL 2: JP 2014-523778 A

### SUMMARY OF INVENTION

### Technical Problem

However, the illness odors-suppressing sheet for medical use described in PTL 1 would be effective in some degree for suppression of odors, but has a problem in that the sheet could not sufficiently absorb the exudates from the area of a lesion. The exudates from a lesion contain odors, and therefore if such exudates leak out from the sheet, they may give off odors to be a problem. In addition, when the exudates from the area of a lesion spread to the peripheral skin and tissue, there occurs another problem in that the area of the lesion is thereby enlarged.

The wound covering material described in PTL 2 has an absorbent layer and therefore can absorb exudates and odors from the area of a lesion, but as a result of investigations made by the present inventors, it has been clarified that the absorbing effect is not sufficient. In particular, sulfurous illness-associated malodors that may be given off by advanced cancer patients would be strong even though a few, and it is desired to improve the sheet so as to attain a sufficient deodorizing effect.

Given the situation, for the purpose of solving the technical problems in the related art, the present inventors have promoted investigations for providing an absorbent sheet that can sufficiently absorb fluids such as exudates and the like to leak from a lesion and can exhibit a powerful deodorizing effect for illness-associated odors.

### Solution to Problem

As a result of assiduous studies made for the purpose of solving the above-mentioned problems, the present inventors have found that an absorbent sheet having an absorbent region that contains cellulosic fibers, an absorbent polymer, and a thermal adhesive resin, and a deodorizing region that contains cellulosic fibers, a deodorizing agent and a thermal adhesive resin can sufficiently absorb exudates exuding from the area of a lesion and can exhibit a powerful deodorizing effect for illness-associated odors. Specifically, the present invention has the following constitution.
[1] An absorbent sheet with deodorizing functions having an absorbent region and a deodorizing region situated to at least one side of the absorbent region, wherein the absorbent region contains cellulosic fibers, an absorbent polymer and a thermal adhesive resin, and the deodorizing region contains cellulosic fibers, a deodorizing agent and a thermal adhesive resin.
[2] The absorbent sheet with deodorizing functions according to [1], wherein the absorbent polymer is absorbent fibers.
[3] The absorbent sheet with deodorizing functions according to [1] or [2], wherein the content of the absorbent polymer is 10 to 50% by mass relative to the total mass of the solid content constituting the absorbent region.
[4] The absorbent sheet with deodorizing functions according to any of [1] to [3], wherein the absorbent polymer contains a sodium polyaerylate resin.
[5] The absorbent sheet with deodorizing functions according to any of [1] to [4], wherein the deodorizing agent contains a component that reacts with at least one selected from sulfides, sulfurs and amines.
[6] The absorbent sheet with deodorizing functions according to any of [1] to [5], wherein the deodorizing agent contains a polyphenol and a polyphenol oxidase.
[7] The absorbent sheet with deodorizing functions according to any of [1] to [5], wherein the deodorizing agent contains at least one selected from metals, minerals, activated carbons and vegetable extracts.
[8] The absorbent sheet with deodorizing functions according to any of [1] to [7], further having a surface protective layer.
[9] The absorbent sheet with deodorizing functions according to any of [1] to [8], further having at least any resin layer selected from a functional resin layer and an adhesive resin layer, between the absorbent region and the deodorizing region.
[10] The absorbent sheet with. deodorizing functions according to any of [I] to [9], further having a surface contact layer.
[11] The absorbent sheet with deodorizing functions according to any of [1] to [10], which is for covering an area to give off illness-associated odors.

### Advantageous Effects of invention

According to the present invention, there can be Obtained an absorbent sheet with deodorizing functions that can sufficiently absorb fluids such as exudates and the like to leak from a lesion and can exhibit a powerful deodorizing effect for illness-associated odors. In addition, the absorbent sheet with deodorizing functions of the present invention can prevent the absorbed exudates from spreading in the planar direction and the thickness direction of the sheet and therefore can prevent the exudates and the like from being brought into contact with the peripheral area around a lesion, and further can prevent the once-absorbed fluids from turning back.

### BRIEF DESCRIPTION OF DRAWINGS

FIG. 1 is a schematic cross-sectional view for explaining a configuration of one example of an absorbent sheet with deodorizing functions of the present invention.

FIG. 2 is a schematic cross-sectional view for explaining a configuration of one example of an absorbent sheet with deodorizing functions of the present invention.

FIG. 3 is a schematic view for explaining a configuration of an apparatus for forming an absorbent sheet with deodorizing functions of the present invention.

### DESCRIPTION OF EMBODIMENTS

The present invention is described in detail hereinunder. The description of the constitutive elements of the invention given hereinunder is for some typical embodiments and examples of the invention, but the invention should not be limited to such embodiments. In this description, the numerical range expressed by the wording “a number to another number” means the range that falls between the former number indicating the lower limit of the range and the latter number indicating the upper limit thereof.

(Absorbent Sheet with Deodorizing Functions)

The present invention relates to an absorbent sheet with deodorizing functions, having an absorbent region and a deodorizing region situated to at least one side of the absorbent region. The absorbent region contains cellulosic fibers, an absorbent polymer and a thermal adhesive resin, and the deodorizing region contains cellulosic fibers, a deodorizing agent and a thermal adhesive resin.

In the absorbent sheet with deodorizing functions of the present invention, the cellulosic fibers and the absorbent polymer are bonded and fixed via at least a part of the thermal adhesive resin. The thermal adhesive resin has a function of melting by heat to bond and fix the components of the sheet.

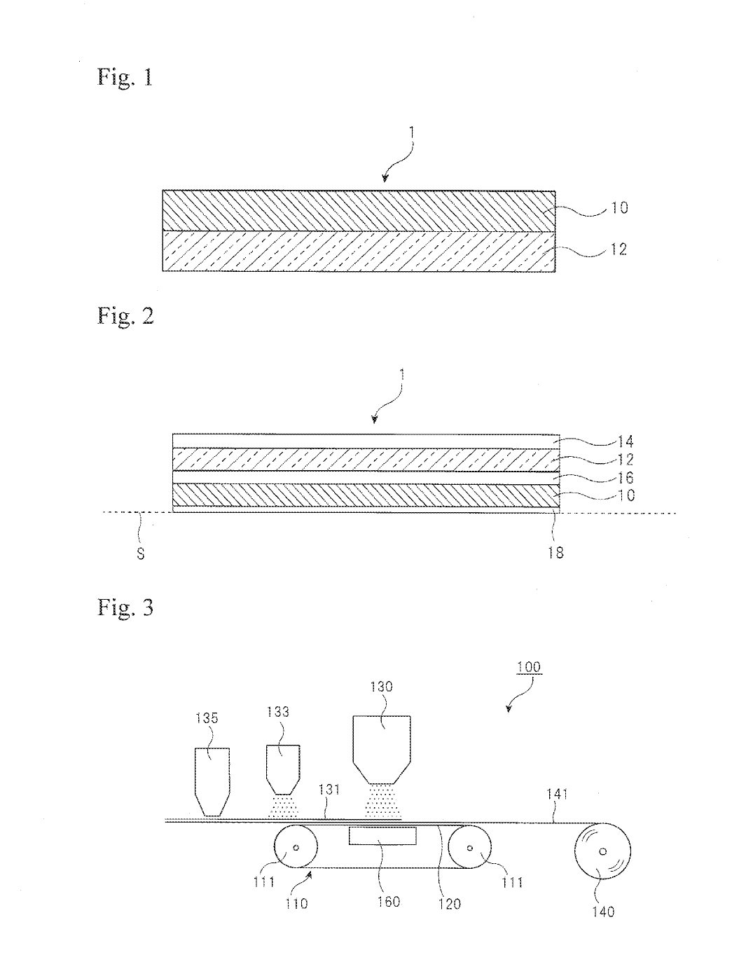

FIG. 1 is a schematic cross-sectional view for explaining a configuration of one example of the absorbent sheet with deodorizing functions of the present invention. As shown in FIG. 1, the absorbent sheet 1 with deodorizing functions of the present invention has an absorbent region 10 and a deodorizing region 12 situated to at least one side of the absorbent region 10. The absorbent sheet 1 with deodorizing functions of the present invention may have plural absorbent regions 10 and plural deodorizing regions 12, but preferably has one absorbent region 10 and one deodorizing region 12. Preferably, the absorbent region 10 and the deodorizing region 12 each have a layered configuration (as sheets) as shown in FIG. 1. In such a case, the absorbent region 10 may be referred to as an absorbent sheet, and the deodorizing region 12 may be referred to as a deodorizing sheet. Specifically, the absorbent sheet with deodorizing functions of the present invention has an absorbent sheet and a deodorizing sheet situated to at least one side of the absorbent sheet. The absorbent sheet with deodorizing functions of the present invention may not have any distinct boundary between the absorbent region 10 and the deodorizing region 12, and the present invention includes such an embodiment.

The absorbent sheet 1 with deodorizing functions of the present invention may not have any distinct boundary between the absorbent region 10 and the deodorizing region 12, but preferably has a configuration where the deodorizing agent contained in the deodorizing region 12 does not move toward the absorbent region 10. For example, a deodorizing agent is made to stably exist in the deodorizing region 12 in order not to drop off or flow away from the deodorizing region 12, or a film or the like capable of blocking passage of a deodorizing agent therethrough is provided between the absorbent region 10 and the deodorizing region 12 to prevent transfer of a deodorizing agent. For example, a resin layer 16 (see FIG. 2) may be provided between the absorbent region 10 and the deodorizing region 12 to thereby prevent a deodorizing agent from dropping off or flowing away from the deodorizing region 12 toward the absorbent region 10.

The absorbent sheet with deodorizing functions of the present invention has an absorbent region and a deodorizing region each composed of specific fibers. and specific components, and therefore can exhibit an excellent fluid (exudate or the like) absorbing performance and a deodorizing effect. The absorbent region enables spot absorption, therefore preventing the absorbed fluids from spreading or turning back.

In the present invention, an absorbent polymer is used in the absorbent region. The morphology of the absorbent polymer is not specifically limited, including, for example, fibers, powders, etc. The absorbent polymer in the form of fibers may be referred to as absorbent fibers, and in the present invention, absorbent fibers are preferably used. Absorbent fibers are composed of an absorbent polymer, and are formed by processing an absorbent polymer into fibers, and the advantages thereof are that the absorbent polymer constituting the absorbent fibers can be prevented from being eccentrically located in the absorbent region and further that, by using absorbent fibers, the absorbent polymer constituting the absorbent fibers can be prevented from dropping off or flowing away from the absorbent region to thereby increase the absorption performance in the absorbent region.

The present invention has another characteristic that the absorbent region has a net-like structure. In the case where absorbent fibers are used as an absorbent polymer, the absorbent fibers are entangled with the cellulosic fibers therein to form a net-like structure. Further, in the case where the thermal adhesive resin is also fibrous, the cellulosic fibers, the absorbent fibers and the thermal adhesive resin fibers are entangled to form a net-like structure. In such a net-like structure, the absorbed fluids can be prevented from spreading in the planar direction and the thickness direction of the absorbent region. Specifically, in the absorbent region, fluids can be spot-like absorbed, and further, the absorbed fluids can be prevented from turning back. In addition, the absorbent region having a net-like structure can exhibit powerful absorption performance not only for fluids having a low viscosity but also for fluids having a high viscosity.

In the invention, the absorbent region having a net-like structure can additionally exhibit a deodorizing function. This may be considered to be because the absorbent fibers absorb malodorous substances and, in addition, the net-like structure itself constituting the absorbent region can trap malodorous substances to exhibit a deodorizing function.

In the absorbent sheet with deodorizing functions of the present invention, the deodorizing region contains a deodorizing agent. Here, some types of deodorizing agents could exhibit a deodorizing function in the presence of water. When such deodorizing agents are used, the boundary between the absorbent region and the deodorizing region is preferably pervious to moisture. In the case where a deodorizing agent can exhibit the function thereof under the condition where presence of water is excluded, a poorly moisture-pervious film may be provided between the absorbent region and the deodorizing region, or a moisture absorbent may be mixed in the absorbent region to thereby prevent moisture penetration.

Further, the present invention is characterized in that the deodorizing agent also contains cellulosic fibers and the deodorizing region also has a net-like structure. In the case where the thermal adhesive resin is fibrous also in the deodorizing region, the cellulosic fibers and the thermal adhesive resin fibers are entangled to form a net-like structure. In the deodorizing region, the deodorizing function is exhibited mainly by the deodorizing agent therein, but the net-like structure itself to constitute the deodorizing region can trap malodorous substances to exhibit a more excellent deodorizing effect.

As described above, the absorbent sheet with deodorizing functions of the present invention can exhibit an excellent fluid absorbing performance and a deodorizing effect. In addition, the sheet can prevent absorbed fluids from spreading and turning back. Consequently, the absorbent sheet with deodorizing functions of the present invention is favorable for covering an area that may give off illness-associated odors. Specifically, the sheet can be used for covering an area of a lesion caused by invasion in the skin from breast cancer, skin cancer, head and neck cancer, etc. In addition, apart from the above--mentioned use, the sheet can also be used for covering an area of bedsores, or as a constituent member for diapers. Further, the sheet can also be used for bedclothes such as bed sheets, etc., and for bag-like storage cases, etc.

The density, the weight, the vapor permeability and the like of the absorbent sheet with deodorizing functions of the present invention are not specifically limited. The density, the weight, the vapor permeability and the like of the absorbent sheet with deodorizing functions of the present invention may be controlled depending on the use and the function thereof.

Preferably, the absorbent region and the deodorizing region are formed according to an air-laid method. Here, the air-laid method is a type of a method for forming a nonwoven fabric in dry. Specifically, according to the method, fibers to constitute each region are opened in dry, and the opened fibers and various components are, while mixed, deposited on a running wire fabric along with air serving as a medium to thereby form a web (nonwoven sheet) thereon. The fibers to constitute the web are bonded to each other according to various bonding methods. In that manner, the absorbent region and the deodorizing region each are preferably a nonwoven fabric formed according to an air-laid method (air-laid nonwoven sheet).

The air-laid nonwoven fabric sheet can be so controlled that the sheet is bulky and the space between the material fibers therein is small. In addition, in the air-laid nonwoven fabric sheet, the fibers are aligned three-dimensionally. Consequently, in the absorbent region, liquid absorption can be enhanced. In addition, in the deodorizing region, the deodorizing efficiency can be increased.

### (Absorbent Region)

The absorbent region is a region containing cellulosic fibers, an absorbent polymer and a thermal adhesive resin. The absorbent region may be a region composed of cellulosic fibers, an absorbent polymer and a thermal adhesive resin. The absorbent region may be a sheet-like one (absorbent sheet).

The extent of absorption in the absorbent region is preferably 1000 g/m² or more, more preferably 1500 g/m² or more, even more preferably 2500 g/m² or more, still more preferably 4000 g/m² or more, and most preferably 5000 g/m² or more. The extent of absorption means an amount of water remaining in the absorbent sheet with deodorizing functions after the sheet is immersed in a physiological saline solution for 10 minutes and then let the sheet drip for 1 minute.

The density and the weight of the absorbent region are not specifically limited. Preferably, the density and the weight of the absorbent region are so controlled that the region could attain a desired extent of absorption. By controlling the density and the weight in accordance with the use and the function thereof, an excellent fluid absorption performance can be attained, and the absorbed fluids can be prevented from spreading in the planar direction and the thickness direction. Further, the absorbent region enables spot-like absorption and can prevent the once absorbed fluids from turning back toward the skin surface, etc., and can additionally exhibit a deodorizing function.

### <Cellulosic Fibers>

As the cellulosic fibers, various cellulosic fibers heretofore used in absorbents can be employed. For example, the material of the cellulosic fibers includes pulp, rayon, cotton, supra, etc. Above all, pulp fibers are preferably used as the cellulosic fibers, and pulp fibers are excellent in point of fiber length, productivity, raw material cost, etc. One type alone or two or more types of cellulose fibers may he used either singly or as combined.

Examples of the pulp fibers include those obtained from raw material pulp such as wood pulp (softwood, hardwood), or non-wood pulp such as rag pulp, linter pulp, liners pulp, kozo (mulberry tree) pulp, mitsumata (paper bush) pulp, ganpi (bark of clove-like bush) pulp, etc.; recycled waste-paper pulp, etc. Depending on the manufacturing method thereof, pulp may be grouped into categories of mechanical pulp (GP, RGP, TMP) and chemical pulp (sulfate pulp, kraft pulp). Above all, wood pulp is preferably used for pulp fibers, as excellent in supply, quality stability, cost, etc.

The mass-average fiber length of the pulp fibers is not specifically limited, but is preferably 0.05 to 5 mm. Having a fiber length falling within the range, the pulp fibers can be prevented from dropping off from the absorbent region and can enhance the fluid absorption efficiency of the region.

The content of the cellulosic fibers contained in the absorbent region is not specifically limited, but is preferably 1 to 80% by mass relative to the total mass of the solid content constituting the absorbent region. Having a content of the cellulosic fibers falling within the range, the fluid absorbability in the absorbent region can be enhanced more. In this description, the total mass of the solid content constituting the absorbent region means the total mass of the solid content constituting the region where the content of the absorbent polymer is 1% by mass or more.

### <Absorbent Polymer>

The absorbent polymer is preferably a superabsorbent polymer (SAP), and “superabsorbent” means that the polymer can absorb water in an amount of 20 times or more the self-weight. The absorbent polymer (SAP) includes starch-type, cellulosic, synthetic resin-type polymers, etc., such as starch-acrylic acid (salt) graft copolymer, isobutylene-maleic acid copolymer, saponified starch-ethyl acrylate graft copolymer, saponified starch-methyl methacrylate graft copolymer, saponified starch-acrylonitrile copolymer, saponified starch-acrylamide graft copolymer, acrylic acid (salt) polymer, acrylic acid-crosslinked polyethylene oxide, crosslinked sodium carboxymethyl cellulose, crosslinked polyvinyl alcohol-maleic anhydride copolymer, amino acid-crosslinked biodegradable polyaspartic acid, culture product from Alcaligenes laws, etc. Above all, acrylic acid (salt) polymer is preferably used, and in particular, sodium polyacrylate-type resin is preferably used. Specifically, the absorbent polymer for use in the present invention preferably contains a sodium polyacrylate resin. The absorbent fibers may be fibrillated fibers or nanofibers, and these may he combined in any desired manner.

In the present invention, absorbent fibers (SAF) are preferably used as the absorbent polymer (SAP). The absorbent fibers (SAF) are those prepared by fibrillating an absorbent polymer (SAP) that absorbs water to swell.

The content of the absorbent polymer contained in the absorbent region is not specifically limited, but is preferably 1 to 70% by mass relative to the total mass of the solid content constituting the absorbent region, more preferably 10 to 50©% by mass. Having a content of the absorbent polymer falling within the range, the fluid absorbability of the absorbent region can be effectively increased. In addition, having a content of the absorbent polymer falling within the range, the region can prevent any unintentional moisture absorption in use environments.

### <Thermal Adhesive Resin>

The thermal adhesive resin is a binder component for binding the cellulosic fibers and the absorbent polymer in the absorbent region. The thermal adhesive resin melts at least partly by heating and then the molten resin solidifies to bind the cellulosic fibers and the absorbent polymer. Examples of the thermal adhesive resin of the type include synthetic fibers of polyethylene (including copolyethylene), polypropylene (including modified polypropylene, and copolypropylene), low-melting-point polyester (for example, low-melting-point polyethylene terephthalate), low-melting-point polyamide, low-melting-point polylactic acid, polybutylene succinate, acrylic resin, urethane resin, styrene-butadiene copolymer, polyvinyl alcohol (PVA), ethylene-vinyl acetate copolymer (EVA), etc.

The thermal adhesive resin may be in the form of fibers of a single resin, or may also be a composite resin of a combination of two or more kinds of resins. For example, a core/sheath type composite resin is preferably used, which is formed of a core part of a resin having a high melting point and a sheath part of a resin having a low melting point and covering the outer periphery of the core part. The composite form of a combination of two types of resins differing in the melting point include PET/PET composite resin, PE/PE composite resin, PP/PP composite resin, EVA/PP composite resin, PE/PET composite resin, PP/PET composite resin, PE/PP composite resin, Above all, a core/sheath type composite resin formed of a core part of polypropylene fibers (inciting point 160° C.) and a cover layer of polyethylene (melting point 130° C.) (PE/PP composite resin) and a core/sheath type composite resin formed of a core part of PET fibers and a cover layer of polyethylene (PE/PET composite resin) are preferably used. In addition, those having crimped fibers are also preferred as readily giving a soft sheet. In the case where such a core/sheath type composite resin is used, preferably, the cover layer alone is melted by applying hot air thereto at a temperature at which the outer cover layer melts but the core part does not melt (as one example, at 140° C.) In this case, the core part does not melt and therefore can remain as stable fibers. Accordingly, the voids are not damaged in melting and bonding, and therefore an absorbent region having a net-like structure can be readily formed.

In addition, in the present invention, a side-by-side type composite resin may also be used, in which two types of resins differing in the melting point are arranged to be adjacent to each other.

The form of the thermal adhesive resin is not specifically limited, and may be any of a powdery, granular or fibrous form or a combination thereof combined in any desired manner. Above all, the thermal adhesive resin is preferably fibrous. Such thermal adhesive resin fibers are favorably used since they have both functions of a thermal adhesive resin and fibers by themselves. The thermal adhesive resin fibers may be crimped, curled or spiraled, or may be fibrillated like pulp fibers.

In the case where the thermal adhesive resin is powdery or granular, the mean particle size thereof is not specifically limited. In the case where the thermal adhesive resin is fibrous, the mass-average fiber length and fiber diameter of the thermal adhesive resin fibers are not also specifically limited, and may he adequately selected within a range capable of exhibiting the advantageous effects of the present invention.

The content of the thermal adhesive resin contained in the absorbent region is not specifically limited, but is preferably 1 to 70% by mass relative to the total mass of the solid content constituting the absorbent region.

### <Other Components>

The absorbent region may be composed of the above-mentioned constituent components alone, but may contain any other components as needed. Examples of the other components include functional powder, functional fibers, functional liquid, etc. The functional powder is preferably one having any one or more functions of antiallergenic function, antiviral function, aromatic function, etc. For example, there are mentioned titanium oxide, titanium dioxide, magnesium oxide, vegetable extract, flavonol, collagen fibers, iron oxide, citric acid, tannic acid, hinokithiol, eucalyptus extract, etc. The functional fibers and the functional liquid may be fibers or liquid containing the above-mentioned functional powder.

### (Deodorizing Region)

The deodorizing region is a region containing cellulosic fibers, a deodorizing agent and a thermal adhesive resin. The deodorizing region may be a region composed of cellulosic fibers, a deodorizing agent and a thermal adhesive resin. The deodorizing region is a region not substantially containing an absorbent polymer. Here, the state not substantially containing an absorbent polymer means a state where the content of an absorbent polymer is less than 1% by mass. The deodorizing region may be a sheet (deodorizing sheet).

The density and the weight of the deodorizing region are not specifically limited. The density and the weight of the deodorizing region are preferably so controlled that the region could exhibit desired deodorizing functions. Accordingly, the region can effectively exhibit the deodorizing effect.

### <Cellulosic Fibers>

As examples of the cellulosic fibers contained in the deodorizing region, those exemplified hereinabove for the cellulosic fibers contained in the absorbent region may be referred to. For preferred fiber types, fiber length and others, those of the cellulosic fibers contained in the absorbent region mentioned hereinabove may be referred to. The cellulosic fibers contained in the deodorizing region and the cellulosic fibers contained in the absorbent region may be the same type or different types.

The content of the cellulosic fibers contained in the deodorizing region is not specifically limited, but is preferably 1 to 80% by mass relative to the total mass of the solid content constituting the deodorizing region. In this description, the total mass of the solid content constituting the deodorizing region means the total mass of the solid content constituting the region where the content of the deodorizing agent is 1% by mass or more.

### <Deodorizing Agent>

The deodorizing agent preferably contains a component capable of reacting with a causative compound that may give off illness-associated odors. Here, examples of the causative compound that may give off illness-associated odors include nitrogen-containing compounds, sulfur-containing compounds, amine compounds, phenols, lower fatty acids, etc. Specifically, the illness-associated odors--emitting causative compound includes, though not limited thereto, trimethylamine, Isovaleric acid, dimethyl trisulfide, methylmercaptan, cadaverine, putrescine, diacetyl, etc. Above all, the deodorizing agent for use in the present invention preferably contains a component capable of reacting with at least one selected from nitrogen-containing compounds and sulfur-containing compounds, and preferably contains a component capable of reacting with at least one selected from sulfides, sulfurs and amines. Further, the deodorizing agent for use in the present invention preferably contains a component capable of reacting with sulfides, above all preferably contains a component capable of reacting with at least one selected from dimethyl trisulfide and methylmercaptan, and diamines such as cadaverine, putrescine, etc. The form of the deodorizing agent is not specifically limited, and the agent may be incorporated as powder or fibers.

Here, “reacting with” the above-mentioned compound means that (a) the deodorizing agent chemically reacts with the illness-associated odors-emitting causative compound to thereby change the illness-associated odors-emitting causative compound into any other having a structure not emitting odors, (b) the deodorizing agent physically absorbs the illness-associated odors-emitting causative compound, (c) the deodorizing agent masks the odors of the illness-associated odors-emitting causative compound. Owing to such “reaction”, the deodorizing agent can exhibit the deodorizing effect. For example, the deodorizing agent that chemically reacts with an illness-associated odors-emitting causative compound includes a deodorizing agent containing a vegetable extract, a polyphenol and a polyphenol oxidase; and the deodorizing agent that physically absorbed an illness-associated odors-emitting causative compound includes metals and minerals, for example, zeolite, silver ion-containing zeolite, etc., as well as activated carbon, etc. The deodorizing agent capable of masking the odors of an illness-associated odors-emitting causative compound includes a deodorizing agent containing an aromatic component. Above all, for illness-associated odors-emitting causative compounds of sulfides, use of a deodorizing agent containing any of metals, minerals, activated carbons, vegetable extracts, polyphenols and polyphenol oxidases is more preferred.

The deodorizing agent may also be one that breaks down microbes such as bacteria, fungi, yeasts and others or prevents such microbes from growing to thereby suppress malodors. The deodorizing agent of the type includes zinc pyrithione, zinc oxide, chitin, chitosan, organic nitrogenous/sulfurous compounds, escallops, and substances having a microbicidal (antimicrobial)/fungicidal (antifungal) function.

In the present invention, use of a deodorizing agent containing at least one selected from metals, minerals, activated carbons and vegetable extracts is preferred. In addition, use of a deodorizing agent containing a polyphenol and a polyphenol oxidase is also preferred. The polyphenol that the deodorizing agent can contain is a compound having two or more hydroxyl groups substituted for the hydrogen atoms on one and the same benzene ring, and a glycoside thereof is also included in the polyphenol. In addition, the polyphenol may be a polymer.

The polyphenol that the deodorizing agent can contain is not specifically limited, but above all, a polyphenol having a hydroquinone or o-diphenol structure is preferred. The o-diphenol structure means a structure where hydroxyl groups directly substituted to a benzene ring and the hydroxyl groups are adjacent to each other.

Examples of polyphenols include apigenin, apigenin glycoside, acacetin, isorhamnetin, isorhamnetin glycoside, isoquercitrin, epicatechin, epicatechin gallate, epigallocatechin, epigallocatechin gallate, aesculetin, ethylprotocatechuate, ellagic acid, catechol, gamma acid, catechin, gardenine, gallocatechin, caffeic acid, caffeate, ehlorogenic acid, kaempferol, kaempferol glycoside, quercetin, quercetin glycoside, quercetagenin, genisetin, genisetin glycoside, gossypetin, gossypetin glycoside, gossypol, 4-dihydroxyanthraquinone, 1,4-dihydroxynaphthalane, cyanidine, cyanidine glycoside, sinensetin, diosmetin, diosmetin glycoside, 3,4′-diphenyldiol, sinapinic acid, stearyl-β-(3,5-di-t-butyl-4-hydroxyphenyl)propionate, spinacetin, tangeretin, taxifolin, tannic acid, daphnetin, tyrosine, delphinidin, delphinidin glycoside, theaflavin, theaflavin monogallate, theaflavin bisgallate, tricetinidin, dopa, dopamine, naringenin, naringin, nordihydroguaiaretic acid, noradrenalin, hydroquinone, vanillin, patchouletin, herbacetin, vanillyl alcohol, vanitrope, vanillin propylene glycol acetal, vanillic acid, bis(4-hydroxyphenyl)sulfonic acid, bisphenol A, pyrocatechol, vitexin, 4,4′-biphenyidiol, 4-t-butylcatechol, 2-tert-butylhydroquinone, protocatechuic acid, phloroglucinol, phenolic resin, procyanidin, prodelphinidin, phloretin, phioretin glycoside, fisetin, folin, ferbacetin, fraxetin, phloridin, peonidin, peonidin glycoside, pelargonidin, pelargonidin glycoside, petunidin, petunidin glycoside, hesperetin, hesperidin, gallic acid, gallate: (lauryl gallate, propyl gallate, butyl gallate), mangeferin, malvidin, malvidin glycoside, myricetin, myricetin glycoside, 2,2′-mehtylenebis(4-methyl-6-t-butylphenol), 2,2′-methylenebis(4-ethyl-6-t-butylphenol), 2,2′-methylenebis(4-methyl-6-tert-butylphenol), methyl atrarate, 4-methylcatechol, 5-methylcatechol, 4-methoxycatechol, 5-methoxycatechol, methylcatechol-4-carboxylic acid, 2-methylresorcinol, 5-methylresorcinol, morin, limocitrin, limocitrin glycoside, limocitrol, luteolin, luteolin glycoside, luteolinidin, luteolinidin glycoside, rutin, resorcin, resveratrol, resorcinol, leucocyanidin, leueodelphinidin, etc. One alone or two or more of these polyphenols may be used either singly or as combined.

The above-mentioned polyphenols may be prepared according to known methods, but commercial products are also available. In addition, they may be prepared through synthesis. Further, high-concentration polyphenol fractions prepared from vegetables can also be used.

Preferred examples of polyphenol oxidase are oxidases contained in vegetable extracts. Specifically, there are mentioned oxidases contained in burdock extract, coffee bean extract, green tea leaf extract, etc. Oxidase contained in burdock extract is a preferred example.

As the deodorizing agent, commercial products can be used. Specifically, DEOATAK (registered trademark) manufactured by Takasago International Corporation and others are usable.

Examples of metals include Cu, Fe, Ni, Zn, Ag, Ti, Co, Al, Cr, Pb, Sn, In, Zr, Mo, Mn, Cd, Bi, Mg, etc. Preferably, the metal exists as metal fine particles and/or metal ions.

Minerals are hydrous silicates with a metal such as aluminum, iron, manganese, magnesium, calcium, potassium, sodium or the like, and are aggregates of fine crystal pieces. Examples of the minerals include smectites (montmorillonite, beidellite, nontronite, saponite, hectorite, sauconite, stevensite, etc.), micas (sericite, illite, muscovite, phlogopite, etc.), vermiculite, chlorite, pyrophyllite, talc, kaolin minerals, serpentinite, sepiolite, allophane, hydrotalcite, etc. In addition, metal oxides such as silicon dioxide, calcium oxide and the like are also usable.

Examples of activated carbon include coconut carbon, coal, wood coal, etc.

Examples of vegetable extracts include extracts extracted with solvent or the like from turmeric, gambir, common sage, tea, rosemary, fennel, thyme, nutmeg, pepper, turmeric, vanilla, bell pepper, coix seed, bupleurum root, Japanese quince, sapanwood, eulen, John love, oxalis, chameleon plant, hemlock, ginkgo, Japanese black pine, larch, Japanese red pine, empress tree, fortune tea olive, lilac, orange osmanthus, sweet coitsfoot, crested leopard, forsythia, chestnut, alder, quercus serrata, pomegranate, fig, flowering fern, weigela, persimmon, fleawort, mugwort, mountain maple, crape myrtle, Lespedeza thunbergii var. albiflora, Japanese andromeda, fern, aspen, Japanese ash, sweet potato (sucrose), eucalyptus, etc.

Apart from vegetable extracts, deodorizing agents derived from organic acids such as amino acids or the like, as well as from natural products, vegetables or food materials are mentioned as vegetable-derived deodorizing agents. For example, cyciodextrin and the like are usable. Regarding cyclodextrin, α-, β- or γ-type ones may be selected or mixed depending on the intended use.

One alone or plural deodorizing agents mentioned above may be used either singly or as combined. In addition, any other deodorizing agents than those mentioned above may also be combined and used. Using plural kinds of deodorizing components could make it possible to cope with various kinds of illness-associated odors-emitting causative mechanisms.

### <Thermal Adhesive Resin>

The thermal adhesive resin is a binder component that binds the cellulosic fibers in the deodorizing region. The thermal adhesive resin is a binder component that makes the above-mentioned deodorizing agent held among the cellulosic resins.

The thermal adhesive resin contained in the deodorizing region includes resins and resin fibers exemplified hereinabove for the thermal adhesive resin contained in the absorbent region. Preferred resin species, fiber length and others are the same as those of the thermal adhesive resin contained in the absorbent region.

Though not specifically limited, the content of the thermal adhesive resin contained in the deodorizing region is preferably 1 to 70% by mass relative to the total mass of the solid content constituting the deodorizing region.

### <Other Components>

The deodorizing region may be composed of the above-mentioned constituent components alone, but may contain any other components as needed. Examples of the other components include functional powder, functional fibers, functional liquid, etc. The functional powder is preferably one having any one or more functions of antiallergenic function, antiviral function, aromatic function, etc. For example, there are mentioned titanium oxide, titanium dioxide, magnesium oxide, vegetable extract, flavonol, collagen fibers, iron oxide, citric acid, tannic acid, hinokithiol, eucalyptus extract, etc. The functional fibers and the functional liquid may be fibers or liquid containing the above-mentioned functional powder.

(Layer Configuration of Absorbent Sheet with Deodorizing Functions)

The absorbent sheet with deodorizing functions of the present invention has an absorbent region and a deodorizing region situated to at least one side of the absorbent region, and preferably further has a surface protective layer. In the absorbent sheet with deodorizing functions of the present invention, the absorbent region is arranged on the side near to a skin surface and the deodorizing region is arranged on one side of the absorbent region and on the side opposite to a skin surface. With that, the surface protective layer is laminated on one side of the deodorizing ion and on the side opposite to the absorbent region.

As shown in FIG. 2, the absorbent sheet 1 with deodorizing functions of the present invention may further have a surface protective layer 14 in addition to the absorbent region 10 and the deodorizing region 12. The surface protective layer 14 may be a nonwoven fabric but is preferably a waterproof film. The surface protective layer 14 of a waterproof film can protect the surface that is an exposed surface during use from wetting with water, etc., and therefore can more effectively exhibit liquid absorbability and deodorizing effect. In addition, exchange frequency of the absorbent sheet with deodorizing functions can be reduced. Further, the waterproof film is preferably pervious to vapor (pervious to moisture) as capable of preventing a lesion from getting stuffy.

The surface protective layer 14 includes a waterproof film, a moisture-pervious film, an unwoven fabric (for example, a water-repellent unwoven fabric), etc. The waterproof film includes a PE film, etc. The nonwoven fabric includes a spunbond nonwoven fabric, a spunlace nonwoven fabric, a thermal bonded nonwoven fabric, SMS (spunbond/melt-blownispunbond) nonwoven fabric, etc.

Further, the absorbent sheet with deodorizing functions of the present invention preferably has at least any resin layer selected from a functional resin layer and an. adhesive resin layer, between the absorbent region and the deodorizing region. As shown in FIG. 2, the absorbent sheet 1 with deodorizing functions of the present invention has an absorbent region 10 and a deodorizing region 12, and preferably further has a resin layer 16. The resin layer 16 may be a single layer of any of a functional resin layer or an adhesive resin layer, and may be a multiple layer formed by laminating a functional resin layer and an adhesive resin layer. For example, preferably, an absorbent region 10, a functional resin layer, an adhesive resin layer and a deodorizing region 12 are laminated in that order from a skin surface side S.

Examples of the functional resin layer that can constitute the resin layer 16 include a polyethylene film, a polyester film, a polyvinyl chloride film, a polycarbonate film, etc.

The adhesive resin layer that can constitute the resin layer 16 is preferably formed of an adhesive resin. Examples of the adhesive resin include a hot-melt adhesive (hereinafter this may be simply referred to as hot-melt). The component to constitute the hot-melt adhesive includes thermoplastic components such as polyvinyl acetate resin, polyvinyl butyral, acrylic resin, polyamide, ethylene-vinyl acetate copolymer, ethylene-acrylate copolymer, etc. (these thermoplastic components include olefinic resin components); natural resin components such as rosin, rosin derivative, etc.; rubber components such as butyl rubber, polyisobutylene, etc. The adhesive is not applied to the entire surface, but is applied partly. Specifically, pattern-like or mesh-like coating is preferred. Examples of the pattern-like coating include slit-like, spiral or dot-like coating.

The resin layer 16, if any, in the absorbent sheet 1 with deodorizing functions is preferably pervious to moisture. In particular, in the case where the deodorizing agent exhibits the deodorizing function⁻in the presence of water, the resin layer 16 preferably has moisture perviousness.

Also preferably, the resin layer 16 has a function of preventing the deodorizing agent contained in the deodorizing region 12 from moving out toward the absorbent region 10. For example, the resin layer 16 may have water repellency.

Preferably, the absorbent sheet with deodorizing functions of the present invention further has a surface contact layer. As shown in FIG. 2, the absorbent sheet 1 with deodorizing functions of the present invention preferably has a surface contact layer 18 on one side of the absorbent region 10 and on the side to be kept in contact with a skin surface S. The surface contact layer 18 is a layer to be in contact with an. illness-associated odors emission area such as a wounded area, a lesion area, etc. Accordingly, the surface contact layer 18 is preferably formed of a material that does not irritate a wounded area and others and that does not stick to a wounded area.

The surface contact layer 18 has a structure capable of transferring fluids and. odors-emitting causative substances such as exudates and the like exuding from a wounded area to the absorbent region 10 and the deodorizing region 12. Specifically, the surface contact layer 18 includes a moisture-pervious film, a nonwoven fabric. (for example, a water-repellent nonwoven fabric), a resin film, a resin net, etc. The nonwoven fabric includes a spunbond nonwoven fabric, a spunlace nonwoven fabric, a thermal bonded nonwoven fabric, SMS (spunbond/melt-blown/spunbond) nonwoven fabric, etc. Examples of the resin film include a PET film, etc. As the resin net, for example, Delnet X550 WHITE-E (manufactured by DelStar Incorporation) and Polynet (manufactured by Smith & Nephew KK) are usable.

In using a resin film, preferably, the resin film is perforated for securing fluid permeability.

The surface contact layer 18 is preferably a non-adhesive layer. When the surface contact layer 18 is a non-adhesive layer, it can prevent the absorbent sheet with deodorizing functions from adhering (sticking) to a wounded area. When the surface contact layer 18 is a non-adhering layer, it reduces irritation to be given to the wounded area when the absorbent sheet with deodorizing functions is peeled from a wounded area.

Preferably, the non-adhering layer is formed, for example, by providing fine pores through the surface contact layer 18. Using a net-like sheet, a perforated film or an embossed sheet as the non-adhering layer is also a preferred embodiment. In such a manner, the structure of the non-adhering layer is preferably so planned that the contact area thereof to a skin surface S is reduced. In addition, fluid permeability is needed so that the absorbent sheet can absorb fluids. Beyond that, as the method for forming the non-adhering layer, a method of incorporating an oily component such as vaseline or the like into the layer, a method of vapor-depositing a component not adhering to skin such as aluminum or the like on the layer, or a method of incorporating or applying silicone to the layer is preferred.

The absorbent sheet with deodorizing functions of the present invention may further have any other layer than the above-mentioned layers. Examples of the other layer include a vapor-pervious carrier sheet, etc. Examples of the vapor-pervious carrier sheet include, though not specifically limited thereto, tissue paper, spunbond fabrics, spunlace fabrics and the like pervious to vapor. The vapor-pervious carrier sheet is, for example, a supporting layer in forming the absorbent region and the deodorizing region, and is used in manufacture. After the sheet formation, the vapor-pervious carrier sheet may be left as such, or may be peeled off. Within a range not detracting the advantageous effects of the present invention in that manner, any layer may be laminated.

In addition, in laminating the above-mentioned layers and regions, if desired, an adhesive layer may be provided between the layers.

The total thickness of the absorbent sheet with deodorizing functions of the present invention is, though not specifically limited thereto, preferably 1 cm or less when a load of 7 gf/cm² is applied thereto. The total thickness of the absorbent sheet with deodorizing functions may be adequately adjusted depending on the use of the absorbent sheet with deodorizing functions.

Method for Producing Absorbent Sheet with Deodorizing Functions)

The present invention relates to a method for producing the above-mentioned. absorbent sheet with deodorizing functions. One preferred embodiment of the method for producing the absorbent sheet with deodorizing functions of the present invention includes a step of mixing cellulosic fibers, an absorbent polymer and a thermal adhesive resin in air or the like and depositing the resultant mixture onto a vapor-pervious carrier sheet to form a sheet for an absorbent region, a step of forming a sheet for a deodorizing region of cellulosic fibers, a thermal adhesive resin and a deodorizing agent, and a step of laminating the sheet for an absorbent region and a sheet for a deodorizing region. In the step of forming a sheet for a deodorizing region, preferably, cellulosic, fibers, a deodorizing agent and a thermal adhesive resin are mixed in air or the like, and the resultant mixture is deposited to form a sheet for a deodorizing region.

In the step of forming a sheet for a deodorizing region, first, cellulosic fibers, an absorbent polymer and a thermal adhesive resin are mixed in air or the like (mixing step). The mixing step is a step of mixing cellulosic fibers, an absorbent polymer and a thermal adhesive resin to prepare an absorbent sheet material. In the present invention, absorbent fibers are preferably used as the absorbent polymer, and in the step for forming a sheet for an absorbent region, preferably, cellulosic fibers, absorbent fibers and a thermal adhesive resin are mixed in air, and deposited to form a sheet for an absorbent region. In mixing the fibers, a stirrer may be used, but preferably, the fibers are mixed using an air stream. In the mixing step, the fibers are preferably opened. By opening the fibers, a bulky sheet can be formed.

The mixture of cellulosic fibers, an absorbent polymer and a thermal adhesive resin is deposited on a vapor-pervious carrier sheet on a wire fabric (vapor-pervious belt) running with a medium of air. FIG. 3 shows a schematic view for explaining a constitution of an apparatus 100 for forming an absorbent sheet with deodorizing functions of the present invention. As shown in FIG. 3, a mixture of cellulosic fibers, an absorbent polymer and a thermal adhesive resin is supplied from a fiber mixture supply unit 130, and deposited on a wire fabric (vapor-pervious belt) 120 to form an air-laid web 131 thereon. The fiber mixture as a web material is three-dimensionally and randomly deposited using an air stream. In that manner, the fibers are deposited to be in a predetermined thickness or more, thereby forming a sheet for an absorbent region (this may also he referred to as a sheet forming step or a web forming step). The sheet forming step is called an air-laid method, and the sheet formed in the air-laid method is called an air-laid web. As a typical process of an air-laid method, for example, a J & J method, a K-C method, a Honshu method (also called a Kinocloth method) and the like are known. In the case where an absorbent polymer is powdery or granular, the polymer may he supplied from the a powder supply unit 133, etc.

In the sheet forming step, for example, the sheet forming apparatus 100 equipped with a conveyor 110, the wire fabric (vapor-pervious belt) 120, the fiber mixture supply unit 130 and a suction box 160 may be used. In the sheet forming apparatus 100 of the type, the fiber mixture supply unit 130 is arranged above the wire fabric (vapor-pervious belt) 120. The wire fabric (vapor-pervious belt) 120 runs, driven by on the conveyor 110 equipped with plural rollers 111. With that, a fiber mixture deposits on the wire fabric (vapor-pervious belt) 120 as assisted by the suction flow from the suction box 160 arranged below the wire fabric (vapor-pervious belt) 120. The fiber mixture drops down along with the air flow and deposits to form an air-laid web.

Above the vapor-pervious belt (wire fabric), a vapor-pervious carrier sheet 141 supplied from a vapor-pervious carrier sheet supply unit 140 is arranged, and the fiber mixture may be deposited on the vapor-pervious carrier sheet 141. Thus arranged, the vapor-pervious carrier sheet 141 enables efficient deposition of the fiber mixture. Examples of the vapor-pervious carrier sheet 141 include tissue paper, thermal bond nonwoven fabric, etc. The vapor-pervious carrier sheet 141 may be peeled off after formation of the absorbent region sheet, or may be left as such without being peeled.

In the ease where a fiber mixture is deposited on the vapor-pervious carrier sheet 141, occasionally, a thermal adhesive resin may be previously sprayed on the vapor-pervious carrier sheet 141. The amount of the thermal adhesive resin to be sprayed is preferably small, and the resin is preferably so sprayed that the amount thereof could be less than the unit weight of the vapor-pervious carrier sheet, more preferably in an amount of less than 10 g/m². By spraying the thermal adhesive resin in a small amount, the liquid permeability can be kept high and the adhesiveness between the vapor-pervious carrier sheet and the fiber mixture sheet can be enhanced.

The web formed in the sheet forming step contains a thermal adhesive resin. Therefore, the formed web is preferably subjected to heat treatment (binding step). The heat treatment may be carried out, for example, using a heat treatment unit 135 shown in FIG. 3. Through the heat treatment, at least a part of the thermal adhesive resin melts and binds the constituent fibers. The heating temperature in the binding step is preferably a temperature at which at least a part of the thermal adhesive resin could melt. The method of melting at least a part of a thermal adhesive resin to bind fibers is called a thermal bonding method. Specifically, in the present invention, the sheet is formed in combination of an air-laid web method and a thermal bonding method. When an air-laid method is combined with a thermal bonding method in bonding fibers, a sheet having a small density and having many pores can be produced, and the sheet is excellent in absorption performance.

The heat treatment method in the binding step includes hot air treatment, infrared irradiation treatment, etc. As an example of hot air treatment, there is mentioned a method of heat treatment where an air-laid web is brought into contact with a through-air drier equipped with a rotary drum having a vapor-pervious periphery (hot air circulation rotary drum system). As another example, there is mentioned a method of heat treatment Where an air-laid web is led into a box type drier in which hot air is applied to the air-laid web for heat treatment (hot air circulation conveyor oven system).

After the binding step, a step of compression treatment using a hot roll or the like may be provided for the purpose of controlling the thickness and the density of the sheet. The formed sheet may be stored after wound up in a roll.

Preferably, the sheet for a deodorizing region is formed according to the same process as that for forming the above-mentioned sheet. In this case, in the step of forming the sheet for a deodorizing region, cellulosic fibers, a deodorizing agent and a thermal adhesive resin may be mixed in air to form a web. Besides, a deodorizing agent may be incorporated from the powder supply unit 133 into the web formed by mixing cellulosic fibers and a thermal adhesive resin, or a deodorizing agent may be further incorporated from the powder supply unit 133 into the web formed by mixing cellulosic fibers, a deodorizing agent and a thermal adhesive resin in air and depositing the mixture. Regarding the method of incorporating a deodorizing agent, there may be further mentioned, though not limited thereto, a method of applying a deodorizing agent to a web by a spray or the like. A sheet for a deodorizing region can be formed according to the method mentioned above, and the thus-formed sheet for a deodorizing region has an excellent deodorizing effect.

The sheet for an absorbent region and the sheet for a deodorizing region produced according to the above-mentioned method are laminated to form an absorbent sheet with deodorizing functions. As the method for laminating the sheet for an absorbent region and the sheet for a deodorizing region, for example, there are three methods mentioned below.

(1) A sheet for an absorbent region (hereinafter a sheet before heat treatment may be read as a web) and a sheet for a deodorizing region are produced in each step, and then the sheets are stuck together.

(2) A roll formed by winding up a sheet for a deodorizing region is previously prepared, and while a sheet for an absorbent region is formed in the step shown in FIG. 3, the sheet for a deodorizing region is stuck thereto. In this case, the heat treatment unit 135 may be provided before sticking the sheet for a deodorizing region, or may be provided after sticking the sheet for a deodorizing region.

(3) A roll formed by winding up a sheet for an absorbent region is previously prepared, and while a sheet for a deodorizing region is formed in the step shown in FIG. 3, the sheet for an absorbent region is stuck thereto. Also in this case, the heat treatment unit 135 may be provided in any stage before or after sticking the sheet for an absorbent region.

The sheet for an absorbent region and the sheet for a deodorizing, region may be bonded via the thermal adhesive resin contained in each sheet, but are preferably laminated via an additional adhesive resin layer provided separately.

In the case where the absorbent sheet with deodorizing functions of the present invention has a surface protective layer, and/or at least any resin layer selected from a functional resin layer and an adhesive resin layer, and/or a surface contact layer, preferably, the layers are separately formed and laminated in a predetermined order. In lamination, an adhesive resin suitable for adhering the layers may be used.

The absorbent sheet with deodorizing functions of the present invention is preferably an absorbent sheet with deodorizing functions produced according to the above-mentioned production method. The absorbent sheet with deodorizing functions of the type can exhibit excellent fluid absorbability and deodorizing performance.

### EXAMPLES

The characteristics of the present invention are further described concretely with reference to Examples given hereinunder. In the following Examples, the material used, its amount and ratio, the details of the treatment and the treatment process may be suitably modified or changed not overstepping the spirit and the scope of the invention. Accordingly, the invention should not be limitatively interpreted by the Examples mentioned below.

### Example 1

### Formation of Sheet for Absorbent Region

The following (a) to (c) were mixed uniformly with an air flow to prepare a fiber mixture for forming an absorbent region.

(a) As cellulosic fibers, pulp fibers (softwood chemical pulp) having a fiber length of 0.05 to 5 mm.

(b) As synthetic fibers, PE/PP core,/sheath type composite fibers of 2.2 dtex×5 mm length.

(c) As superabsorbent fibers (SAF), sodium polyacrylate superabsorbent fibers having a fiber length of 6 mm arid a fiber diameter of 10 dtex,

The above (a), (b) and (c) were contained in the fiber mixture so as to be in an amount of 136 g/m², 57 g/m² and 61 g/m², respectively, in the air-laid web (sheet for absorbent region) to be formed.

Next, on a vapor-pervious belt running as mounted on a conveyor, a vapor-pervious carrier sheet of a thermal bond nonwoven fabric having a unit weight of 22 g/m² was supplied by a vapor-pervious carrier sheet supply unit, and while the vapor-pervious belt was sucked by a suction box, a mixture of the above-mentioned fiber mixture from the fiber mixture supply unit and 4 g/m² of a thermal adhesive synthetic resin (polyethylene powder) was dropped down along with an air flow and deposited thereon to form an air-laid web. The air-laid web was heated at 140° C. in a heating furnace, and further, from another carrier sheet supply unit, a carrier sheet of a moisture-pervious white film (polyethylene film) having a unit weight of 32 g/m² as a resin layer was supplied while pattern-like coated with a hot melt to produce a laminate. Next, the laminate was treated with a pressure roll to give an absorbent region-containing laminate sheet having a unit weight of 317 g/m² and a thickness of 4 mm.

### Formation of Sheet for Deodorizing Region

Next, the following (a) to (c) were uniformly mixed with an air flow to prepare a fiber mixture for forming a deodorizing region.

(a) As cellulosic fibers, pulp fibers (softwood chemical pulp) having a fiber length of 0.05 to 5 mm.

(b) As synthetic fibers, PE/PP core/sheath type composite fibers of 2.2 dtex×5 mm length.

(c) As a deodorizing agent, zeolite,

Next, on a vapor-pervious belt running as mounted on a conveyor, a vapor-pervious carrier sheet of tissue paper having a unit weight of 14 g/m² was supplied by a vapor-pervious carrier sheet supply unit as a liner, and using a powder feeder, 2 g/m² of a thermal adhesive synthetic resin (polyethylene powder) was sprayed thereon.

Next, while the vapor-pervious belt was sucked by a suction box, the fiber mixture was dropped down along with an air flow from the fiber mixture supply unit and deposited on the vapor-pervious carrier sheet to form an air-laid web. The air-laid web was heated at 140° C. in a heating furnace, and from another carrier sheet supply unit, a carrier sheet of a waterproof film having a unit weight of 30 g/m² as a surface protective layer was supplied while pattern-like coated with a hot melt to produce a laminate. Next, the laminate was treated with a pressure roll to give a deodorizing region-containing laminate sheet having a unit weight of 160 g/m² and a thickness of 2 mm.

Formation of Absorbent Sheet with Deodorizing Functions

The absorbent region-containing laminate sheet was supplied to the back surface of the vapor-pervious carrier sheet (tissue) of the deodorizing region-containing laminate sheet while a hot melt was pattern-like applied thereto, thereby preparing a laminate. The absorbent region-containing laminate sheet was so laminated that the moisture-pervious white film (polyethylene film) of the vapor-pervious carrier sheet (tissue) of the deodorizing region-containing laminate sheet could bond to each other via the pattern-like hot melt therebetween,

Next, the laminate was treated with a pressure roll to give an absorbent sheet with deodorizing functions. The layer configuration of the absorbent sheet with deodorizing functions obtained in Example 1 is composed of, as configured in that order from the face remotest from a skin surface, a waterproof film (surface protective layer of the deodorizing region-containing laminate sheet), a pattern-like hot melt layer, a deodorizing agent-containing layer (deodorizing region), tissue (supporting layer of the deodorizing region), a pattern-like hot melt layer, a polyethylene film (resin layer of the deodorizing region-containing laminate sheet), a pattern-like hot melt layer, a SAF-containing layer (absorbent region), a thermal bond nonwoven fabric (surface contact layer of the deodorizing region-containing laminate sheet).

### Example 2

An absorbent sheet with deodorizing functions was produced in the same manner as in Example 1, except that the deodorizing agent was changed to Mizukanite HP (manufactured by Mizusawa Industrial Chemicals, Ltd.), and further a biaxially-stretched PET film having a thickness of 6 μm was laminated on the side of the vapor-pervious carrier sheet formed of a thermal bond nonwoven fabric of the laminate sheet while perforating the film with hot needles, thereby forming a non-adhesive layer of the surface contact layer.

### Example 3

An absorbent sheet with deodorizing functions was produced in the same manner as in Example 2, except that the deodorizing agent was changed to Taiko CW350BR (manufactured by Futamura Chemical Co., Ltd.) of crushed activated carbon.

### Example 4

An absorbent sheet with deodorizing functions was produced in the same manner as in Example 2, except that the deodorizing agent was changed to Taiko SG840A (manufactured by Futamura Chemical Co., Ltd.) of granular activated carbon.

### Example 5

An absorbent sheet with deodorizing functions was produced in the same manner as in Example 2, except that the deodorizing agent was changed to Shoe Cleanse KD-411G (manufactured by Rasa Industries, Ltd.),

### Example 6

An absorbent sheet with deodorizing functions was produced in the same manner as in Example 2, except that the deodorizing agent was changed to a mixture (mixing ratio 1/1) of Mizukanite HP (manufactured by Mizusawa Industrial Chemicals, Ltd.) and Shoe Cleanse KD-411G (manufactured by Rasa Industries, Ltd.).

### Comparative Example 1

An absorbent sheet was produced in the same manner as in Example 2, except that it did not contain a deodorizing agent.

### (Evaluation)

### <Absorption Amount Measurement Test>

The sheet produced in Examples and Comparative Examples was kept put in a container containing physiological saline in an amount that the sheet could be sufficiently immersed therein, for 10 minutes. Subsequently, the sheet was left hung for making water drip therefrom for 1 minute, and the sheet weight after immersion was measured. The sheet weight before and after immersion was measured, and the absorption amount per the unit area was calculated. The results are shown in Table 1.

### <Deodorizing Test 1>

A deodorizing test was carried out according to the method defined in the certification standards for SEK mark textile products certification standards (Japan Textile Evaluation Technology Council). As odorous components, 5 components of ammonia, acetic acid, acetaldehyde, methylmercaptan and trimethylamine gas were used, and the sample was tested according to an instrumental analysis (detector tube method) test.

### Instrumental Analysis Test:

An odorous component and a sample of an absorbent sheet with deodorizing functions were put in a container, and in 2 hours, the remaining concentration of the odorous component was measured. The remaining concentration in the container with an odorous component alone was measured as a blank test concentration, and the decrease rate of the odorous component was calculated according to the following equation, and the tested sample was evaluated according to the following evaluation standards. The results are shown in Table 1.

Odorous Component Decrease Rate (%)=(blank test concentration at each time−sample test concentration at each time)/(blank test concentration at each time)×100
A: The odorous component decrease rate was 75% or more.
B: The odorous component decrease rates was 50% or more and less than 75%.
C: The odorous component decrease rates was 20% or more and less than 50%.
D: The odorous component decrease rate was less than 20%.

### <Deodorizing Test 2>

A deodorizing test with dimethyl trisulfide was carried out according to the following process. 10 L of air was put into a Tedlar bag, and 10 μL of dimethyl trisulfide was injected thereinto to prepare a gas having a concentration of about 2 ppm. A sample of an absorbent sheet with deodorizing functions having a size of 10 cm square was put into a separate Tedlar bag and sealed up. 10 L of the gas was injected into the Tedlar bag having therein the sample of an absorbent sheet with deodorizing functions, and after a predetermined period of time, the concentration in the bag was measured according to a detector tube method. With that, the odorous component decrease rate was calculated according to the following equation, and the sample was evaluated according to the following evaluation standards. The results are shown in Table 1.

Dimethyl Trisulfide Decrease Rate (%)=(blank test concentration at each time−sample test concentration at each time)/(blank test concentration at each time)×100
A: The dimethyl sulfide decrease rate was 75% or more.
B: The dimethyl sulfide decrease rates was 50% or more and less than 75%.
C: The dimethyl sulfide decrease rates was 20% or more and less than 50%.
D: The dimethyl sulfide decrease rate was less than 20%.

TABLE 1

| | Absorption Amount | Odorous Component Decrease Rate | | | | | |
|---|---|---|---|---|---|---|---|
| | (g/cm²) | Ammonia | Acetic acid | Acetaldehyde | Methylmercaptan | Trimethylamine | Dimethyl sulfide |
| Example 1 | 6600 | A | A | | C | A | |
| Example 2 | 6600 | A | A | | C | A | |
| Example 3 | 6500 | A | A | A | A | A | |
| Example 4 | 6500 | A | A | A | A | A | |
| Example 5 | 7400 | A | A | B | B | A | A |
| Example 6 | 6600 | A | A | B | B | A | A |
| Comparative | 6300 | A | A | D | D | B | D |
| Example 1 | | | | | | | |

The sheets obtained in Examples were confirmed to have sufficient absorbability. In addition, the sheets obtained in Examples were also confirmed to have a deodorizing effect for a variety of odorous components. Further, the sheets obtained in Examples were found to exhibit a powerful deodorizing effect for illness-associated odors and a sufficient absorbability for blood, body fluids, etc.

### REFERENCE SIGNS LIST

1 Absorbent Sheets with Deodorizing Functions
10 Absorbent Region
12 Deodorizing Region
14 Surface Protective Layer
16 Resin Layer
18 Surface Contact Layer
S Skin Surface
100 Sheet Forming Apparatus
110 Conveyor
111 Roller
120 Wire Fabric (vapor-pervious belt)
130 Fiber Mixture Supply Unit
133 Powder Supply Unit
131 Air-Laid Web
135 Heat Treatment Unit
140 Vapor-Pervious Carrier Sheet Supply Unit
141 Vapor-Pervious Carrier Sheet
160 Suction Box

1. An absorbent sheet with deodorizing functions having an absorbent region and a deodorizing region situated to at least one side of the absorbent region, wherein:
the absorbent region contains cellulosic fibers, an absorbent polymer and a thermal adhesive resin, and
the deodorizing region contains cellulosic fibers, a deodorizing agent and a thermal adhesive resin.

2. The absorbent sheet with deodorizing functions according to claim 1, wherein the absorbent polymer is absorbent fibers.

3. The absorbent sheet with deodorizing functions according to claim 1, wherein the content of the absorbent polymer is 10 to 50% by mass relative to the total mass of the solid content constituting the absorbent region.

4. The absorbent sheet with deodorizing functions according to claim 1, wherein the absorbent polymer contains a sodium polyacrylate resin.

5. The absorbent sheet with deodorizing functions according to claim 1, wherein the deodorizing agent contains a component that reacts with at least one selected from sulfides, sulfurs and amines.

6. The absorbent sheet with deodorizing functions according to claim 1, wherein the deodorizing agent contains a polyphenol and a polyphenol oxidase.

7. The absorbent sheet with deodorizing functions according to claim 1, wherein the deodorizing agent contains at least one selected from metals, minerals, activated carbons and vegetable extracts.

8. The absorbent sheet with deodorizing functions according to claim 1, further having a surface protective layer.

9. The absorbent sheet with deodorizing functions according to claim 1, further having at least any resin layer selected from a functional resin layer and an adhesive resin layer, between the absorbent region and the deodorizing region.

10. The absorbent sheet with deodorizing functions according to claim 1, further having a surface contact layer.

11. The absorbent sheet with deodorizing functions according to claim 1, which is for covering an area to give off illness-associated odors.
